(19) United States
(12) Patent Application Publication
(10) Pub. No.: US 2012/0183958 A1
(43) Pub. Date: Jul. 19, 2012

(54) METHODS FOR PREDICTING FAT AND LEAN PHENOTYPES IN CHICKENS

(75) Inventor: Larry A. Cogburn, New London, PA (US)

(73) Assignee: University of Delaware, Newark, DE (US)

(21) Appl. No.: 13/008,466

(22) Filed: Jan. 18, 2011

(51) Int. Cl. C12Q 1/68

(52) U.S. Cl. 435/6.11

(57) ABSTRACT

The invention provides molecular methods for predicting chickens that are more likely to have a lean phenotype, comprising detecting in samples of genetic material obtained from the chickens for the presence of paired single nucleotide polymorphisms (SNPs) in the beta-defensin 9 (DEFB9) gene.

### REFERENCE TO U.S. GOVERNMENT SUPPORT

This work is supported by a grant from USDA-IFAFS, Animal Genome Program (Award Number 00-52100-9614). The United States has certain rights in the invention.

### FIELD OF THE INVENTION

The present invention relates generally to methods for predicting fat and lean phenotypes in chickens based on genetic polymorphisms associated with fatness or leanness traits. More specifically, the invention relates to paired single nucleotide polymorphisms (SNPs) in the chicken beta-defensin 9 (DEFB9) gene which are associated with heritable fatness traits, for example, abdominal fat weight (AF) and abdominal fatness as a percentage of body weight (ABFP), and methods for predicting chickens that are more likely to have a fat or lean phenotype by detecting the paired SNPs in the chicken DEFB9 gene.

### BACKGROUND OF THE INVENTION

Excessive accumulation of abdominal fat in broiler chickens is a serious issue faced by the global poultry industry because of economic losses due to lower lean carcass yield, reduced feed efficiency and rejection of fatty meat by concerned consumers (Gaya et al., 2006, Poult Sci. 85(5):837-43). A high heritability estimate (0.53) for abdominal fat content in broiler chickens suggests that this trait would respond to selection (Gaya et al., 2006, Poult Sci. 85(5):837-43). In order to decipher the metabolism and genetic mechanisms involved in the regulation of fatness in the chicken, some investigators have developed experimental models of adiposity. Lean and fat chicken lines have been divergently selected for low or high abdominal fat (Leclercq et al., 1980, Br. Poul. Sci. 21: 107-113) and for very low density lipoprotein (VLDL) plasma concentration (Whitehead and Griffin, 1984, Br. Poult, Sci. 25: 573-582). Studies performed in lean and fat lines developed by Leclercq et al (1980) indicate that the difference in adiposity between lines was not the result of a difference in food consumption or in metabolic utilization. Stearoyl-Co-A desaturase activity and plasma VLDL concentration were found to be higher in the fat line (Legrand and Hermier, 1992, Int. J. Obesity 16: 289-294), suggesting a higher lipogenesis rate in this line. In chickens, lipogenesis occurs essentially in liver and adipose tissues are only storage tissues (O'Hea and Leveille, 1968, Comp. Biochem. Physiol. 26, 111-120. 1968; Griffin et al., 1992, J. Nutri. 122, 363-368).

Fatness is a polygenic trait in chickens controlled by a number of different loci and multiple genes with additive effects. Several quantitative trait locus (QTL) analyses have shown multiple QTL for fatness in chickens (Lagarrigue et al., 2006, Genet. Sel. Evol. 38:85-97; Abasht et al., 2006, Genet. Sel. Evol. 38:297-311). Two independent genome-wide screens of the chicken genome sequence and clustered chicken EST sequences have identified a single highly conserved cluster of β-defensin genes on GGA3 (Lynn et al., 2004, Immunogenetics 56:170-7; Xiao et al., 2004, BMC Genomics 5:56). The β-defensin genes, formerly called gallinacins, in chickens (Lynn et al., 2007, Immunol. Lett. 110:86-9, encode a family of antimicrobial peptides involved in innate immune responses, primarily in the gastrointestinal and reproductive tracts (Hasenstein et al., 2006, Infect. Immun. 74:3375-80; Milona et al., 2007, Biochem. Biophys. Res. Commun. 356:169-74).

The expression of beta-defensin 9 (DEFB9) gene, a member of the β-defensin gene family, appears to be involved in adipogenesis as its expression is up-regulated in the liver of genetically fat chickens, hypothyroid slightly-obese chickens (Cogburn et al., 2003, Poult. Sci. 82:939-51; Wang et al., 2007, Cytogenet. Genome Res. 117:174-88) and in chickens with corticosterone-induced obesity (Hall et al, 2006, FASEB J. 20:A523-a), whereas hepatic expression of DEFB9 is down-regulated by hyperthyroidism (Cogburn et al., 2003, Poult. Sci. 82:939-51; Wang et al., 2007, Cytogenet. Genome Res. 117:174-88) which reduces body fatness. The differential expression of the DEFB9 gene in the liver of chickens under experimental states of leanness or fatness led to the discovery of a pair of linked single nucleotide polymorphisms (SNPs) in several cDNA clones sequenced from chicken liver cDNA libraries. The consensus cDNA sequence (UD Contig_{—}25151.1) derived from the alignment of cDNA sequences (EST clones) is identical to the chicken DEFB9 gene located on the distal end of chromosome 3 (GGA3). Methods for identifying a fat or lean phenotype in chickens have been developed by detecting the SNPs in the single-stranded DEFB9 DNA from the chickens (U.S. patent application Ser. No. 10/376,120, Pub. No. 2007009909). Genotyping a single-stranded RNA or DNA from individual chickens is labor extensive. Thus, there remains a need for commercially more feasible and more cost effective methods for predicting chickens that are more likely to have a fat or lean phenotype based on the presence of functional polymorphisms associated with a fat or lean chicken phenotype in chicken genetic materials that can be easily prepared.

### SUMMARY OF THE INVENTION

The present invention provides an improved method for predicting a chicken that is more likely to have a lean phenotype. The method comprises detecting in a sample of genetic material obtained from the chicken for the presence of paired single nucleotide polymorphisms at positions corresponding to nucleotides 258 (SNP1) and 294 (SNP2) in the beta-defensin 9 (DEFB9) cDNA sequence (SEQ ID NO: 1) (SNP1_SNP2 genotype). The DEFB9 SNP1_SNP2 genotype related to leanness is CC_CC.

The invention also provides an improved method for predicting a chicken that is more likely to have a fat phenotype. The method comprises detecting in a sample of genetic material obtained from the chicken for the presence of paired single nucleotide polymorphisms at positions corresponding to nucleotides 258 (SNP1) and 294 (SNP2) in the beta-defensin 9 (DEFB9) cDNA sequence (SEQ ID NO: 1) (SNP1_SNP2 genotype). The SNP1_SNP2 genotype is selected from the group consisting of CC_TT, TT_CC and CT_CT.

The genetic material comprises double-stranded DNA, preferably genomic DNA. The sample may comprise cells, tissues, blood or bodily fluid, and preferably whole blood, obtained from a chicken embryo or hatched chicken at different ages.

### BRIEF DESCRIPTION OF THE DRAWINGS

FIGS. 1A-D show the cDNA contig sequence for the chicken β-defensin 9 (DEFB9) gene (cDNA Contig_{—}25151.1) (SEQ ID NO: 1), the genomic sequence of the chicken DEFB9 gene on chromosome 3 (SEQ ID NO: 2), and the alignment of the DEFB9 cDNA contig and genomic sequences. Matching bases in cDNA and genomic sequences are capitalized. Underlined bases mark the boundaries of gaps in either sequence. Aligned blocks with gaps≦8 bases are merged for the display when only one sequence has a gap, or when gaps in both sequences are of the same size.

FIG. 2 shows the detailed alignment of 16 partial chicken expressed sequence tags (ESTs) having two single nucleotide polymorphisms at positions corresponding to nucleotides (nt) 258 (SNP1) and 294 (SNP2) of the cDNA contig sequence (SEQ ID NO: 1) of the chicken DEFB9 gene, also known as Gallinacin-9 (Gal9).

### DETAILED DESCRIPTION OF THE INVENTION

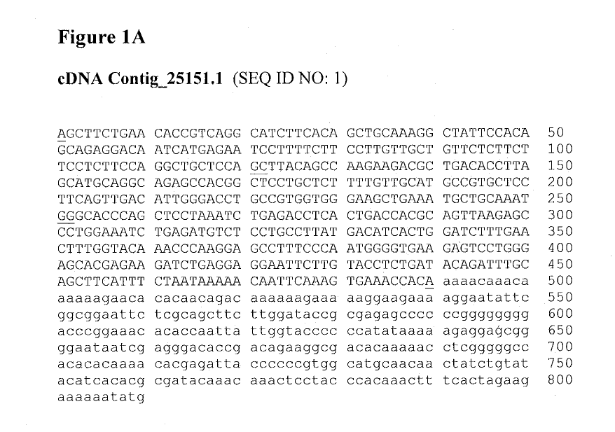

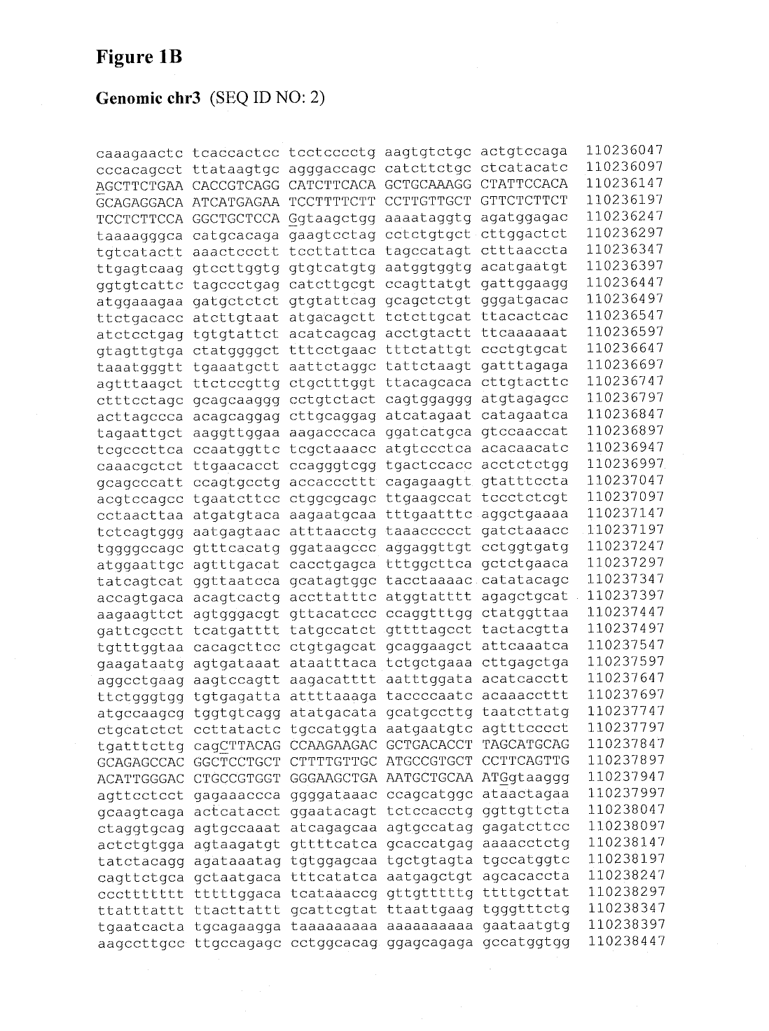

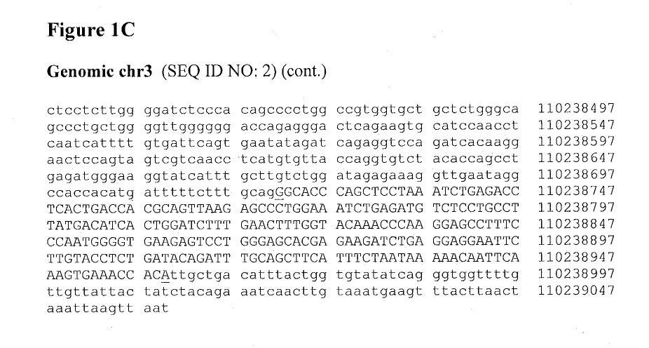

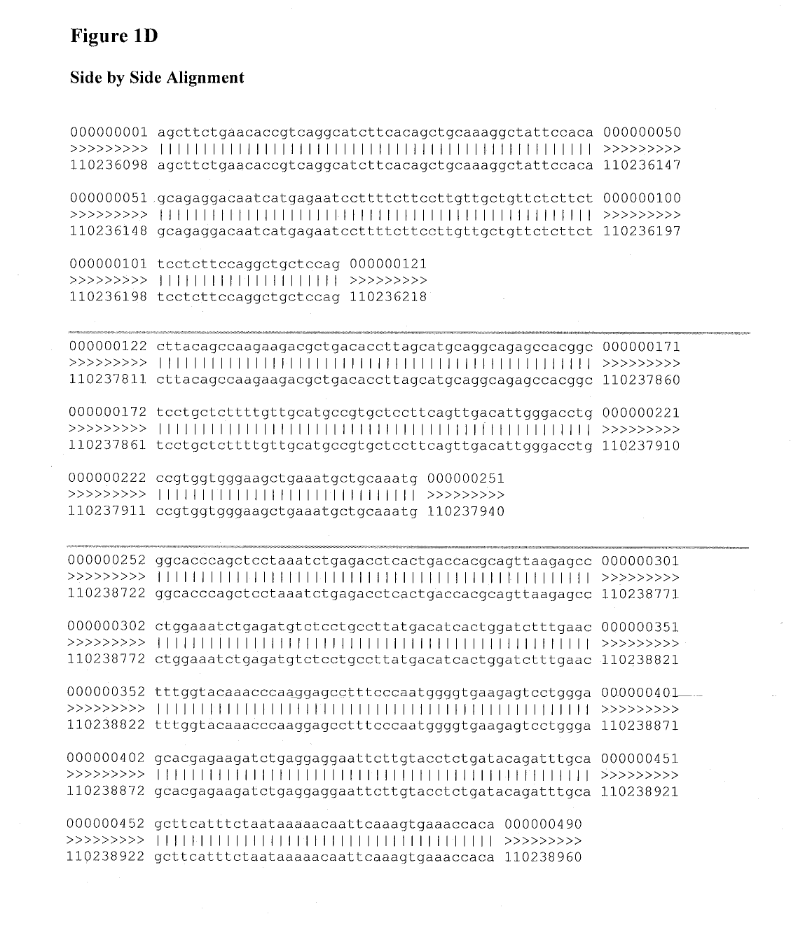

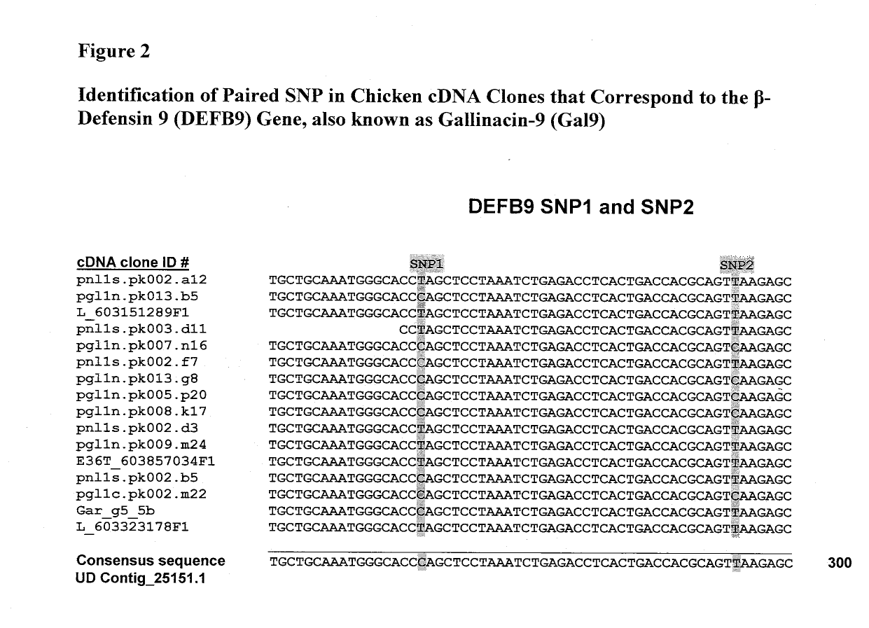

Two single nucleotide polymorphisms, SNP1 and SNP2, were previously found in the chicken β-defensin 9 (DEFB9) gene (FIGS. 1 and 2). The chicken DEFB9 gene is located on chromosome 3 at nucleotides 110,236,098 to 110,238,960 in the UCSC Genome Browser on Chicken May 2006 (Build WUGSC 2.1/galGal3) Assembly (see alignment in FIG. 1). The SNP1 site is located at position 195 relative to the first base of the start codon of the DEFB9 gene, corresponding to nucleotide 258 in the DEFB9 cDNA sequence (SEQ ID NO: 1) and nucleotide 110,238,728 of the DEFB9 genomic sequence (SEQ ID NO: 2). The SNP2 site is located at position 231 relative to the first base of the start codon of the DEFB9 gene, corresponding to nucleotide 294 in the DEFB9 cDNA sequence (SEQ ID NO: 1) and nucleotide 110,238,764 of the DEFB9 genomic sequence (SEQ ID NO: 2). Specific genotypes at the SNP1 and SNP2 sites in single stranded DEFB9 DNA were previously identified to be associated with a fat or lean phenotype in chickens, and used in screening for chickens that are more likely to have a fat or lean phenotype.

The present invention is based on the new discovery that chickens having specific paired polymorphisms at the SNP1 site (corresponding to nucleotide 258 in SEQ ID NO: 1 or nucleotide 110,238,728 of SEQ ID NO: 2) and at the SNP2 site (corresponding to nucleotide 294 in SEQ ID NO: 1 or nucleotide 110,238,764 in SEQ ID NO: 2) (SNP1_SNP2 genotype) in double-stranded DEFB9 DNA tend to have a fat or lean phenotype. These DEFB9 alleles can be used as molecular markers for predicting leanness and fatness in chickens and genetic selection for leanness in poultry breeding programs. Improved screening methods according to the present invention involve determination of the DEFB9 SNP1_SNP2 genotype in double-stranded DEFB9 DNA from chickens.

The degree of leanness or fatness selected in a given population of chickens could vary depending on the genetic background and the selection criteria. A fat or lean phenotype may be determined based on a phenotypic difference in one of the heritable fatness traits, for example, abdominal fat weight (AF) and abdominal fatness as a percentage of body weight (ABFP). The difference may be statistically significant.

In one embodiment, chickens having homologous CC at the SNP 1 and SNP2 sites, SNP1_SNP2 genotype of CC_CC, tend to have a lean phenotype. Improved methods for predicting chickens that are more likely to have a lean phenotype comprise detecting in samples of genetic material obtained from the chickens for the presence of the CC_CC genotype at the DEFB9 SNP1_SNP2 locus. This CC_CC genotype at the DEFB9 SNP1_SNP2 locus is a genetic marker for leanness.

In another embodiment, chickens having genotypes of CC_TT, TT_CC or CT_CT at the DEFB9 SNP1_SNP2 locus, or fat markers, tend to have a fat phenotype. Improved methods for predicting chickens that are more likely to have a fat phenotype comprise detecting in samples of genetic material obtained from the chickens for the presence of a SNP1_SNP2 genotype of CC_TT, TT_CC or CT_CT. The CC_TT, TT_CC or CT_CT genotype at the DEFB9 SNP1_SNP2 locus is a genetic marker for fatness.

The methods according to the present invention may further comprise obtaining samples of genetic material from chickens at different ages, including chicken embryos. The genetic material may be isolated from cells, tissues, blood or other samples according to standard methodologies. In certain embodiments, analysis is performed on whole cell or tissue homogenates or biological fluid samples without substantial purification of the template nucleic acid. The genetic material comprises double-stranded DNA, preferably genomic DNA. A preferred source of genetic material is whole blood. Chickens have nucleated red blood cells That makes blood a convenient source of genetic material (genomic DNA). Blood samples can be obtained from chickens at different ages after hatching (e.g., newly hatched chicks, juvenile birds, and adult birds), or from the embryo even before hatching. The chickens may be breeding stocks.

The detecting step may be carried out on a blood sample from a chicken embryo in an egg to predict whether the embryo has the leanness marker (or genotype of CC_CC at the DEFB9 SNP1_SNP2 locus) and whether a chicken if hatched from the egg will likely to have a lean phenotype. An embryo having the leanness marker may be allowed to hatch in order to improve body composition (increased leanness).

Chickens having the leanness marker (or genotype of CC_CC at the DEFB9 SNP1_SNP2 locus) may be used for breeding to produce lean offspring.

The polymorphisms indicative of a fat or lean phenotype can be identified by any method known in the art for detection of alleles at specific polymorphic sites. Suitable methods include sequencing the genetic material, polymerase chain reaction (PCR)-based assays, primer extension, allele-specific oligonucleotide ligation, high throughput next-generation DNA sequencing and high-density SNP microarray hybridization.

A number of template dependent processes are available to amplify the oligonucleotide sequences present in a given template sample. One of the best-known amplification methods is the polymerase chain reaction (referred to as PCR) which is described in detail in U.S. Pat. Nos. 4,683,195, 4,683,202 and 4,800,159, each of which is incorporated herein by reference in its entirety.

The amplification product may be detected or quantified. In certain applications, the detection may be performed by visual means. Alternatively, the detection may involve indirect identification of the product via chemiluminescence, radioactive scintigraphy of incorporated radiolabel or detection of a fluorescent label.

A preferred method for detecting the SNPs is real-time quantitative PCR using dual labeled TaqMan® probes which have a fluorophore at the 5′ end and a quencher at the 3′ end. Methods for performing PCR using dual labeled probes are disclosed in U.S. Pat. Nos. 5,210,015, 5,804,375, 5,487,792 and 6,214,979.

PCR technology relies on thermal strand separation followed by thermal dissociation. During this process, at least one primer per strand, cycling equipment, high reaction temperatures and specific thermostable enzymes are used (U.S. Pat. Nos. 4,683,195 and 4,883,202). Alternatively, it is possible to amplify the DNA at a constant temperature (Kievits et al., 1991, J. Virol Methods 35:273-286; U.S. Pat. No. 5,130,238; EP0500224 A2; Walker et al., 1992, Nuc. Acids Res., 20:1691-1696). Any sequencing method known to a person skilled in the art may be employed. In particular, it is advantageous to use an automated DNA sequencer. The sequencing is preferably carried out with a double-stranded template by means of the chain-termination method using fluorescent primers. An appropriate kit for this purpose is provided from PE Applied Biosystems (PE Applied Biosystems, Norwalk, Conn., USA).

Alternatively, the DNA chip method can be employed (Barinaga M., 1991, Science 253:1489; Bains, W., 1992; Bio/Technology 10:757-758; Wang et al., 1998, Science 280:1077-1082). These methods usually attach specific DNA sequences to very small specific areas of a solid support, such as micro-wells of a DNA chip. Each type of polymorphic DNA of the present invention can be used for the DNA chip when they are hybridized with the amplified DNA fragment of the genetic material sample, and then detected by the pattern of hybridization.

The polymorphisms can also be identified by hybridization to nucleic acid arrays, some examples of which are described in WO 95/11995. The same arrays or different arrays can be used for analysis of characterized polymorphisms. WO 95/11995 also describes subarrays that are optimized for detection of a variant form of a precharacterized polymorphism. Such a subarray contains probes designed to be complementary to a second reference sequence, which is an allelic variant of the first reference sequence. The second group of probes is designed by the same principles as described, except that the probes exhibit complementarity to the second reference sequence. The inclusion of a second group (or further groups) can be particularly useful for analyzing short subsequences of the primary reference sequence in which multiple mutations are expected to occur within a short distance commensurate with the length of the probes (e.g., two or more mutations within 9 to 21 bases).

Amplification products generated using the polymerase chain reaction can be analyzed by use of denaturing gradient gel electrophoresis. Different alleles can be identified based on the different sequence-dependent melting properties and electrophoretic migration of DNA in solution. Erlich, ed., PCR Technology, Principles and Applications for DNA Amplification, (W. H. Freeman and Co, New York, 1992), Chapter 7.

An alternative method for identifying and analyzing polymorphisms is based on single-base extension (SBE) of a fluorescently-labeled primer coupled with fluorescence resonance energy transfer (FRET) between the label of the added base and the label of the primer. Typically, the method, such as that described by Chen et al., 1997, Proc. Nat. Acad. Sci. 94:10756-61, uses a locus-specific oligonucleotide primer labeled on the 5′ terminus with 5-carboxyfluorescein (FAM). This labeled primer is designed so that the 3′ end is immediately adjacent to the polymorphic site of interest. The labeled primer is hybridized to the locus, and single base extension of the labeled primer is performed with fluorescently-labeled dideoxyribonucleotides (ddNTPs). An increase in fluorescence of the added ddNTP in response to excitation at the wavelength of the labeled primer is used to infer the identity of the added nucleotide. Other suitable methods will be readily apparent to the skilled artisan.

The invention also provides primers and probes for use in the assays to detect the SNPs. The primers and probes are based on and selected from SEQ ID NO: 1 and will typically span the region of SEQ ID NO: 1 upstream or downstream of a SNP in the case of primers, or span a SNP site in the case of a probe and will have a length appropriate for the particular detection method. The primers and/or probes can also be based on and selected from the genomic DNA sequence of DEFB9 (SEQ ID NO: 2). One aspect of the invention thus provides oligonucleotides comprising from about 10 to about 30 contiguous bases of SEQ ID NO: 1 or SEQ ID NO: 2, or the complementary sequence of SEQ ID NO: 1 or SEQ ID NO: 2 for use as probes or primers.

Probes can be any length suitable for specific hybridization to the target nucleic acid sequence. The most appropriate length of the probe may vary depending upon the hybridization method in which it is being used; for example, particular lengths may be more appropriate for use in microfabricated arrays (microarrays), while other lengths may be more suitable for use in classical hybridization methods. Such optimizations are known to the skilled artisan. Suitable probes can range from about 5 nucleotides to about 30 nucleotides in length. For example, the probes can be 5, 6, 8, 10, 12, 14, 16, 18, 20, 22, 24, 25, 26, 28 or 30 nucleotides in length. Additionally, a probe can be a genomic fragment that can range in size from about 25 to about 2,500 nucleotides in length. The probe preferably overlaps at least one polymorphic site occupied by any of the possible variant nucleotides. The nucleotide sequence of the probe can correspond to the coding sequence of the allele or to the complement of the coding sequence of the allele.

Preferably, the PCR probes are TaqMan® probes which are labeled at the 5′ end with a fluorophore, and at the 3′-end with a quencher or a minor groove binder and a quencher (for minor groove binding assays). Suitable fluorophores and quenchers for use with TaqMan® probes are disclosed in U.S. Pat. Nos. 5,210,015, 5,804,375, 5,487,792 and 6,214,979.

An oligonucleotide primer can be synthesized by selecting any continuous 10 to 30 base sequence from the DEFB9 cDNA sequence (SEQ ID NO: 1) or genomic sequence (SEQ ID NO: 2), or the complementary sequence thereof. The length of these oligonucleotide primers are commonly in the range of 10 to 30 nucleotides in length, preferably in the range of 18 to 25 nucleotides in length.

Hybridizations can be performed under stringent conditions, e.g., at a salt concentration of no more than 1 M and a temperature of at least 25° C. For example, conditions of 5×SSPE (750 mM NaCl, 50 mM Na-Phosphate, 5 mM EDTA, pH 7.4) and a temperature of 25-30° C., or equivalent conditions, are suitable for allele-specific probe hybridizations. Equivalent conditions can be determined by varying one or more of the parameters given as an example, as known in the art, while maintaining a similar degree of identity or similarity between the target nucleotide sequence and the primer or probe used.

The reaction mixture for amplifying the DNA comprises 4 deoxynucleotide phosphates (dATP, dGTP, dCTP, dTTP) and heat stable DNA polymerase (such as Taq polymerase), which are all known to the skilled person in the art.

The genomic DEFB9 DNA sequence contains introns near to the SNP1 site (FIG. 1). Depending on the methods used to detect the SNPs, it may be necessary to use different primer/probe sets to detect the SNPs in genomic DNA.

Because of the close proximity of the SNPs in the DEFB9 nucleotide sequence, it is necessary, when detecting the SNPs using PCR and dual labeled TaqMan® probes, to detect each SNP separately. The preferred primer/probe sets thus contain a set of primers and probes to detect the polymorphism at SNP1, and a set of primers and probes to detect the polymorphism at SNP2. The preferred primer and probe sets are described in more detail in the examples.

The oligonucleotide primers and probes can be synthesized by any technique known to a person skilled in the art, based on the structure of SEQ ID NO: 1 or SEQ ID NO: 2.

The invention further provides kits comprising at least one allele-specific oligonucleotide or gene expression product indicator as described herein. Often, the kits contain one or more pairs of allele-specific oligonucleotides hybridizing to different forms of a polymorphism. Examples of suitable allele-specific oligonucleotides include the oligonucleotide probes disclosed herein. The kits can also comprise primers for amplifying a region of SEQ ID NO: 1 or SEQ ID NO: 2 that spans a polymorphism. Optionally, the allele-specific oligonucleotides are provided immobilized to a substrate. The assay kit can further comprise the four deoxynucleotide phosphates (dATP, dGTP, dCTP, dTTP) and an effective amount of a nucleic acid polymerizing enzyme. A number of enzymes are known in the art which are useful as polymerizing agents. These include, but are not limited to E. coli DNA polymerase I, Klenow fragment, bacteriophage T7 RNA polymerase, reverse transcriptase, and polymerases derived from thermophilic bacteria, such as Thermus aquaticus. The latter polymerases are known for their high temperature stability, and include, for example, the Taq DNA polymerase I. Other enzymes such as Ribonuclease H can be included in the assay kit for regenerating the template DNA. Other optional additional components of the kit include, for example, means used to label the probe and/or primer (such as a fluorophore, quencher, chromogen, etc.), and the appropriate buffers for reverse transcription, PCR, or hybridization reactions. Usually, the kit also contains instructions for carrying out the methods.

All patents and patent applications cited in the present application are expressly incorporated herein by reference for all purposes. The above disclosure generally describes the present invention. A more complete understanding can be obtained by reference to the following specific examples, which are provided for purposes of illustration only and are not intended to limit the scope of the invention.

### Example 1

### Identification of Paired SNPs in the Chicken Beta-Defensin 9 (DEFB9) Gene

The chickens studied were an F2 population generated from reciprocal crosses between genetically fat and lean chickens developed by Leclercq et al., 1980, Br. Poul. Sci. 21, 107-113. Five cocks from the fat line (FL) were mated with 14 hens from the lean line (LL) resulting in F1 chickens (FL×LL), of which three cocks and twenty six hens were chosen to generate a total number of 339 F2 chickens. For the inverse cross, four cocks from the lean line (LL) were mated with eight hens from the fat line (FL) to generate F1 (LL×FL), of which two cocks and seventeen hens were chosen to produce a total number of 229 F2 individuals. From these 568 F2 individuals, 554 birds passed the pedigree verification check. Therefore, the F2 population used in the association analyses included a total number of 554 F2 chickens with records of number of genotypes (from 129 anonymous microsatellite markers) and 14 phenotypes (quantitative production traits).

The infomativeness (heterogenetity) of the pair of SNPs previously identified in the DEFB9 gene was determined in the grandparents (F0) of genetically fat (FL) and lean (LL) chickens and their the F1 from the FL×LL intercross by DNA sequencing of a PCR amplified fragment containing both SNPs. This sequence information and allele frequency was used to design a custom TaqMan® SNP genotyping assay (Part #4332077; Applied Biosystems, Foster City, Calif.) for high-throughput screening of the paired SNP in DEFB9 in genomic DNA from 554 individuals from the FL×LL F2 resource population.

The target genomic DNA fragment was amplified in a PCR reaction using an ABI Prism Sequence Detection System 7900HT (Applied Biosystems Inc, Foster City, Calif.). The total reaction volume was 5 μl containing 1 μl of 100 ng of DNA (phenol/chloroform purified), 0.125 μl of 40×TaqMan® primers/probe mix, 2.5 μl of 2×TaqMan® Universal PCR Master Mix and 1,375 μl of DNase-free water. Thermal cycle condition was incubation for 2 min at 50° C., 10 min at 95° C. and followed by 40 cycles of 15 sec at 95° C. and 1 min at 60° C. The end product of the PCR reaction was subjected to allelic discrimination analysis using SDS 2.2.1 software in the ABI Prism Sequence Detection System 7900HT. The custom Taqman® SNP genotyping assay and using ABI SDS 2.2.1 software allows determination of the CC, CT and TT genotype at the SNP1 and SNP loci in the chicken DEFB9 gene. The following primers and reporters (or probes) were used in the genotyping assay:

| | |
|---|---|
| Forward Primer Name | Forward Primer Seq. |
| DEFB9_SNP1F | GCTTGTCTGGATAGAGAAAGGTTGA |
| | (SEQ ID NO: 3) |
| Reverse Primer Name | Reverse Primer Seq. |
| DEFB9_SNP1R | GTGGTCAGTGAGGTCTCAGATT |
| | (SEQ ID NO: 4) |
| Reporter 1 Name | Reporter 1 Dye |
| DEFB9_SNP1V2 | VIC |
| Reporter 1 Sequence | Reporter 1 Quencher |
| TAGGAGCTGGGTGCCC | NFQ |
| (SEQ ID NO: 5) | |
| Reporter 2 Name | Reporter 2 Dye |
| DEFB9_SNP1M2 | FAM |
| Reporter 2 Sequence | Reporter 2 Quencher |
| TAGGAGCTAGGTGCCC | NFQ |
| (SEQ ID NO: 6) | |
| Forward Primer Name | Forward Primer Seq. |
| DEFB9_SNP2F | GCTCCTAAATCTGAGACCTCACTGA |
| | (SEQ ID NO: 7) |
| Reverse Primer Name | Reverse Primer Seq. |
| DEFB9_SNP2R | GGCAGGAGACATCTCAGATTTCC |
| | (SEQ ID NO: 8) |
| Reporter 1 Name | Reporter 1 Dye |
| DEFB9_SNP2V2 | VIC |
| Reporter 1 Sequence | Reporter 1 Quencher |
| AGGGCTCTTGACTGCGT | NFQ |
| (SEQ ID NO: 9) | |
| Reporter 2 Name | Reporter 2 Dye |
| DEFB9_SNP2M2 | FAM |
| Reporter 2 Sequence | Reporter 2 Quencher |
| AGGGCTCTTAACTGCGT | NFQ |
| (SEQ ID NO: 10) | |

Association of the polymorphisms and abdominal fat weight (grams) was statistically determined using PROC GLM procedure on SAS v.9.1 (Statistical Analysis System, Cary, N.C.). Stepwise elimination of non-significant effects was employed. The statistical model included the fixed effect of sire, hatching, sex, genotype, and a covariate of individual slaughter weight according to the formula below. Results are shown in Tables 1 and 2.

Yᵢⱼₖₗₘ=μ+Sireᵢ+Hatchingⱼ+Sexₖ+DEFB9ₗ+b(slaughter weight)+eᵢⱼₖₗₘ

### Where:

Sire (i=1-5)
Hatching (j=1-5)
Sex (k=1-2)
DEFB9 genotype (l=1-6)
Slaughter weight as covariate

TABLE 1
Summary of the association statistical analysis

| Effect | P-value |
|---|---|
| Slaughter weight at 8 weeks of age | <0.0001 |
| Sire | <0.0001 |
| Hatching | <0.0007 |
| Sex | <0.0001 |
| SNP1_SNP2 genotype | <0.0123 |

TABLE 2
Association of DEFB9 SNP1_SNP2 genotypes with
fatness traits in the FL × LL F₂ population

| SNP1_SNP2 | Abdominal | Abdominal |
|---|---|---|
| Genotype | fat weight (g)¹ | fat percent² |
| CC_CC | 66.04 ± 3.34^{a} | 2.94 ± 0.184^{a} |
| CC_CT | 72.41 ± 1.70^{a,b} | 3.29 ± 0.126^{a,b} |
| CC_TT | 77.14 ± 2.63^{b,c} | 3.51 ± 0.157^{b,c} |
| CT_CC | 72.05 ± 1.69^{a,b} | 3.28 ± 0.125^{a,b} |
| CT_CT | 76.61 ± 1.35^{b,c} | 3.45 ± 0.116^{b,c} |
| TT_CC | 76.88 ± 1.98^{b,c} | 3.50 ± 0.134^{b,c} |

¹Abdominal fat weight (AF) trait is presented as the least square estimate (LSE) ± standard error of the mean (SEM).
²Abdominal fat percent trait (ABFP) is calculated as abdominal fat as a percent of body weight (% BW), and is also presented as the LSE ± SEM. LSEs possessing different superscripts are significantly different.

The statistical analysis shows a highly significant effect (P<0.01) of the DEFB9 SNP1_SNP2 genotype on abdominal fatness traits (AF and ABFP) in chickens. Genotyping of genomic DNA samples from 554 F2 chickens from the FL×LL cross shows that the homogeneous CC_CC genotype at the SNP1_SNP2 locus represents the leanest phenotype (AF=66 g; ABFP=2.94% BW). The fattest chickens carry one of the following genotypes: CC_TT, TT_CC or CT_CT at the SNP1_{—}2 locus (Table 2), which on average results in a 16.4% increase in abdominal fat weight (or an 18.6% increase in ABFP) when compared to that of the leanest genotype (CC_CC). Thus, there is a difference of about 10.8 g of visceral fat content between the homozygous CC_CC genotype and the CC_TT, TT_CC or CT_CT genotype in this population of chickens. The presence of an homologous CC at one SNP locus in combination with a CT at the other locus (i.e., the CT_CC or CC_CT genotype) increases the abdominal fat weight by about 6.2 g, although the phenotypes (AF or ABFP) of these genotypes were not significantly different from either the leanest (CC_CC) or the fattest (CC_TT, TT_CC or CT_CT) genotypes.

What is claimed:

1. A method for predicting a chicken that is more likely to have a lean phenotype, comprising detecting in a sample of genetic material obtained from the chicken for the presence of paired single nucleotide polymorphisms at positions corresponding to nucleotides 258 (SNP1) and 294 (SNP2) of SEQ ID NO: 1 (SNP1_SNP2 genotype), wherein the SNP1_SNP2 genotype is CC_CC.

2. The method of claim 1, wherein the genetic material comprises double-stranded DNA.

3. The method of claim 1, wherein the genetic material comprises genomic DNA.

4. The method of claim 1, wherein the sample is a whole blood sample.

5. The method of claim 1, wherein the chicken is a chicken embryo.

6. The method of claim 1, wherein the chicken is a hatched chicken.

7. A method for predicting a chicken that is more likely to have a fat phenotype, comprising detecting in a sample of genetic material obtained from the chicken for the presence of paired single nucleotide polymorphisms at positions corresponding to nucleotides 258 (SNP1) and 294 (SNP2) of SEQ ID NO: 1 (SNP1_SNP2 genotype), wherein the SNP1_SNP2 genotype is selected from the group consisting of CC_TT, CT_CT and CC_TT.

8. The method of claim 7, wherein the genetic material comprises double-stranded DNA.

9. The method of claim 7, wherein the genetic material is genomic DNA.

10. The method of claim 7, wherein the sample is a whole blood sample.

11. The method of claim 7, wherein the chicken is a chicken embryo.

12. The method of claim 7, wherein the chicken is a hatched chicken.


SEQUENCE LISTING (10 sequences)

SEQ ID NO: 1 - DNA, 810 bp, Gallus gallus
agcttctgaacaccgtcaggcatcttcacagctgcaaaggctattccacagcagaggacaatcatgagaatccttttcttccttgttgctgttctcttcttcctcttccaggctgctccagcttacagccaagaagacgctgacaccttagcatgcaggcagagccacggctcctgctcttttgttgcatgccgtgctccttcagttgacattgggacctgccgtggtgggaagctgaaatgctgcaaatgggcacccagctcctaaatctgagacctcactgaccacgcagttaagagccctggaaatctgagatgtctcctgccttatgacatcactggatctttgaactttggtacaaacccaaggagcctttcccaatggggtgaagagtcctgggagcacgagaagatctgaggaggaattcttgtacctctgatacagatttgcagcttcatttctaataaaaacaattcaaagtgaaaccacaaaaacaaacaaaaaagaacacacaacagacaaaaaagaaaaaggaagaaaaggaatattcggcggaattctcgcagcttcttggataccgcgagagccccccggggggggacccggaaacacaccaattattggtaccccccatataaaaagaggagcggggaataatcgagggacaccgacagaaggcgacacaaaaacctcgggggccacacacaaaacacgagattaccccccgtggcatgcaacaactatctgtatacatcacacgcgatacaaacaaactcctacccacaaactttcactagaagaaaaaatatg

SEQ ID NO: 2 - DNA, 3063 bp, Gallus gallus
caaagaactctcaccactcctcctcccctgaagtgtctgcactgtccagacccacagcctttataagtgcagggaccagccatcttctgcctcatacatcagcttctgaacaccgtcaggcatcttcacagctgcaaaggctattccacagcagaggacaatcatgagaatccttttcttccttgttgctgttctcttcttcctcttccaggctgctccaggtaagctggaaaataggtgagatggagactaaaagggcacatgcacagagaagtcctagcctctgtgctcttggactcttgtcatacttaaactccctttccttattcatagccatagtctttaacctattgagtcaaggtccttggtggtgtcatgtgaatggtggtgacatgaatgtggtgtcattctagccctgagcatcttgcgtccagttatgtgattggaaggatggaaagaagatgctctctgtgtattcaggcagctctgtgggatgacacttctgacaccatcttgtaatatgacagctttctcttgcatttacactcacatctcctgagtgtgtattctacatcagcagacctgtacttttcaaaaaatgtagttgtgactatggggcttttcctgaactttctattgtccctgtgcattaaatgggtttgaaatgcttaattctaggctattctaagtgatttagagaagtttaagctttctccgttgctgctttggtttacagcacacttgtacttcctttcctagcgcagcaagggcctgtctactcagtggagggatgtagagccacttagcccaacagcaggagcttgcaggagatcatagaatcatagaatcatagaattgctaaggttggaaaagacccacaggatcatgcagtccaaccattcgcccttcaccaatggttctcgctaaaccatgtccctcaacacaacatccaaacgctctttgaacacctccagggtcggtgactccaccacctctctgggcagcccattccagtgcctgaccaccctttcagagaagttgtatttcctaacgtccagcctgaatcttccctggcgcagcttgaagccattccctctcgtcctaacttaaatgatgtacaaagaatgcaatttgaatttcaggctgaaaatctcagtgggaatgagtaacatttaacctgtaaaccccctgatctaaacctggggccagcgtttcacatgggataagcccaggaggttgtcctggtgatgatggaattgcagtttgacatcacctgagcatttggcttcagctctgaacatatcagtcatggttaatccagcatagtggctacctaaaaccatatacagcaccagtgacaacagtcactgaccttatttcatggtattttagagctgcataagaagttctagtgggacgtgttacatcccccaggtttggctatggttaagattcgcctttcatgatttttatgccatctgttttagccttactacgttatgtttggtaacacagcttccctgtgagcatgcaggaagctattcaaatcagaagataatgagtgataaatataatttacatctgctgaaacttgagctgaaggcctgaagaagtccagttaagacattttaatttggataacatcaccttttctgggtggtgtgagattaattttaaagataccccaatcacaaacctttatgccaagcgtggtgtcaggatatgacatagcatgccttgtaatcttatgctgcatctctccttatactctgccatggtaaatgaatgtcagtttccccttgatttcttgcagcttacagccaagaagacgctgacaccttagcatgcaggcagagccacggctcctgctcttttgttgcatgccgtgctccttcagttgacattgggacctgccgtggtgggaagctgaaatgctgcaaatggtaagggagttcctcctgagaaacccaggggataaacccagcatggcataactagaagcaagtcagaactcatacctggaatacagttctccacctgggttgttctactaggtgcagagtgccaaatatcagagcaaagtgccataggagatcttccactctgtggaagtaagatgtgttttcatcagcaccatgagaaaacctctgtatctacaggagataaatagtgtggagcaatgctgtagtatgccatggtccagttctgcagctaatgacatttcatatcaaatgagctgtagcacacctacccttttttttttttggacatcataaaccggttgtttttgttttgcttatttatttatttttacttatttgcattcgtatttaattgaagtgggtttctgtgaatcactatgcagaaggataaaaaaaaaaaaaaaaaaagaataatgtgaagccttgccttgccagagccctggcacagggagcagagagccatggtggctcctcttggggatctcccacagcccctggccgtggtgctgctctgggcagccctgctggggttggggggaccagagggactcagaagtgcatccaacctcaatcattttgtgattcagtgaatatagatcagaggtccagatcacaaggaactccagtagtcgtcaacctcatgtgttaccaggtgtctacaccagcctgagatgggaaggtatcatttgcttgtctggatagagaaaggttgaataggccaccacatgatttttctttgcagggcacccagctcctaaatctgagacctcactgaccacgcagttaagagccctggaaatctgagatgtctcctgccttatgacatcactggatctttgaactttggtacaaacccaaggagcctttcccaatggggtgaagagtcctgggagcacgagaagatctgaggaggaattcttgtacctctgatacagatttgcagcttcatttctaataaaaacaattcaaagtgaaaccacattgctgacatttactggtgtatatcagggtggttttgttgttattactatctacagaaatcaacttgtaaatgaagtttacttaactaaattaagttaat

SEQ ID NO: 3 - DNA, 25 bp, Artificial Sequence
  Synthetic
gcttgtctggatagagaaaggttga

SEQ ID NO: 4 - DNA, 22 bp, Artificial Sequence
  Synthetic
gtggtcagtgaggtctcagatt

SEQ ID NO: 5 - DNA, 16 bp, Artificial Sequence
  Synthetic
taggagctgggtgccc

SEQ ID NO: 6 - DNA, 16 bp, Artificial Sequence
  Synthetic
taggagctaggtgccc

SEQ ID NO: 7 - DNA, 25 bp, Artificial Sequence
  Synthetic
gctcctaaatctgagacctcactga

SEQ ID NO: 8 - DNA, 23 bp, Artificial Sequence
  Synthetic
ggcaggagacatctcagatttcc

SEQ ID NO: 9 - DNA, 17 bp, Artificial Sequence
  Synthetic
agggctcttgactgcgt

SEQ ID NO: 10 - DNA, 17 bp, Artificial Sequence
  Synthetic
agggctcttaactgcgt